(19) United States
(12) Patent Application Publication
(10) Pub. No.: US 2010/0154081 A1
(43) Pub. Date: Jun. 17, 2010

(54) METHODS AND MEANS FOR PRODUCING GLYCOPROTEINS WITH ALTERED GLYCOSYLATION PATTERN IN HIGHER PLANTS

(75) Inventors: Koen Weterings, Raleigh, NC (US); Gerben Van Eldik, Zwijnaarde (BE); Bicke Nagels, Grembergen (BE)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: BAYER BIOSCIENCE N.V., GENT (BE)

(21) Appl. No.: 12/601,131

(22) PCT Filed: May 20, 2008
(86) PCT No.: PCT/EP08/04049

§ 371 Date: Nov. 20, 2009

Related U.S. Application Data
(60) Provisional application No. 60/939,596, filed on May 22, 2007.

(30) Foreign Application Priority Data
May 21, 2007 (EP) 07010060.7

(51) Int. Cl. A01H 5/00; C12P 21/00; C12N 5/04

(52) U.S. Cl. 800/298; 435/69.1; 435/69.6; 435/419

(57) ABSTRACT

The invention provides methods to modify the N-glycosylation pattern of glycoproteins in higher plant cells, through reducing or eliminating the level of β(1,2) xylosyltransferase and α (1,3) fucosyltransferase activity and increasing the β(1,4) galactosyltransferase activity in the cell of the higher plant.

### FIELD OF THE INVENTION

The current invention relates to the field of molecular farming, i.e. the use of plant cells or plants as bioreactors to produce biopharmaceuticals, particularly polypeptides and proteins with pharmaceutical interest such as therapeutic proteins, which have an altered glycosylation pattern that resembles mammalian glycosylation. The invention may also be applied to alter the glycosylation pattern of proteins in plants for any purpose, including modulating the activity or half-life of endogenous plant proteins or proteins introduced in plant cells.

### BACKGROUND

The plant-specific N-glycosylation pathway appears to be one of the major drawbacks impeding the use of recombinant glycoproteins, particularly human glycoproteins, produced in plant cells or plants for therapeutic purposes.

The most important differences between the N-glycosylation pattern in plant glycoproteins and mammalian glycoproteins are the presence of β(1,2) xylosyl and core-α (1,3) fucosyl residues, and the absence of terminal β(1,4) galactosyl residues and sialic acid. β(1,2) xylosyl and core-α (1,3) fucosyl residues are thus absent in humans, and administration of plant-made therapeutical glycoproteins to humans, particularly over longer period could lead to immunogenic or allergic reactions. Moreover, the absence of terminal β(1,4) galactose in plant-made antibodies appears to result in a less efficient immune response than when using a corresponding antibody produced in mammalian cell cultures.

To mimic the N-glycosylation pattern of mammalian glycoproteins in plant-made glycoproteins, the level of the enzymes responsible for incorporation of β(1,2) xylosyl and core-α (1,3) fucosyl residues (i.e. β(1,2) xylosyltransferase (“XylT”) and α (1,3) fucosyltransferase (“FucT”)) needs to be reduced or eliminated in the host plant cells. In addition, the host plant cells need to be supplemented with β(1,4) galactosyltransferase (“GalT”) to achieve N-terminal incorporation of β(1,4) galactosyl in glycoproteins.

EP 1151109 describes the identification of core α (1,3) fucosyltransferase genes from Arabidopsis.

EP 1263968 describes the identification of β(1,2) xylosyltransferase gene from Arabidopsis.

Patent application PCT/EP2007/002322, published as WO2007/107296 describes the identification of β(1,2) xylosyltransferase genes from Nicotiana species.

Cox et al. (2006) (Nature BioTechnology 24, 1591-1597) report glycan optimization of a human monoclonal antibody in Lemnaceae by co-expressing the heavy and light chains of the mAbs with an RNA interference construct targeting expression of the endogenous α (1,3) fucosyltransferase and β(1,2) xylosyltransferase genes.

Koprivova et al. (2004) (Plant Biotechnology Journal 2, 517-523) describe the generation of targeted knockouts of Physcomitrella patens (a moss) wherein both the α (1,3) fucosyltransferase and β(1,2) xylosyltransferase genes were disrupted. The N-glycans of endogenous glycoproteins of these mutant moss plants lacked α (1,3) fucosyl and β(1,2) xylosyl residues. In addition, no such residues were detected on secreted human glycosylated growth factor expressed in such moss cells.

Strasser et al. (2004) (FEBS Letters 561, 132-136) report on the generation of Arabidopsis thaliana knock-out plants which completely lack both XylT and FucT activity. To this end, a triple knock-out plant was generated carrying knock-out mutations in the β(1,2) xylosyltransferase gene as well as in both genes encoding α (1,3) fucosyltransferase (FucTA and FucTB) present in Arabidopsis thaliana. Analysis of the N-glycans in such plants revealed the complete absence of α (1,3) fucosyl and β(1,2) xylosyl residues. Furthermore, less N-glycan heterogeneity was observed in the triple knock-out plants than in wild-type plants, and a high proportion of complex N-glycans carried terminal β N-acetylglucosamine residues on both the α1,3- and α1,6-linked mannoses.

Expression of β(1,4) galactosyltransferase (GalT) in plants has also been reported.

WO00/34490 provides a method for manufacturing a glycoprotein having a human-type sugar chain comprising a step in which a transformed plant cell is obtained by introducing into a plant cell a gene of a glycosyltransferase such as GalT and the gene of an exogenous glycoprotein, and a step in which the obtained transformed plant cell is cultivated.

WO02/057468 describes a method for the secretory production of a glycoprotein having a human-type sugar chain, comprising a step of introducing a gene of an enzyme capable of performing a transfer reaction of a galactose residue to a non-reducing terminal acetyl-glucosamine residue, and a gene of a heterologous glycoprotein, to obtain a transformed plant cells, a step of culturing the plant cell and a step of recovering the culture medium of the plant cell.

WO01/31405 describes a plant comprising a functional mammalian enzyme providing N-glycan biosynthesis that is normally not present in plants (such as a galactosyltransferase) said plant comprising additionally at least a second mammalian protein or functional fragment thereof that is normally not present in plants.

WO01/29242 describes a process for the production of proteins or polypeptides using genetically manipulated plants or plant cells, as well as genetically manipulated plants and plant cells per se. The described plants include transgenic plants comprising Mouse GalT, Bovine GalT or Human GalT.

WO03/078637 describes methods for optimizing glycan processing in organisms (and in particular plants) so that a glycoprotein having complex bi-antennary glycans and thus containing galacose residues on both arms and which are devoid of (or reduced in) xylose and fucose can be obtained.

Palacpac et al. (1999) (Proc. Natl. Acad. Sci. USA 96, 4692-4697) describes stable expression of human β(1,4) galactosyltransferase in plant cells, and the ensuing modified N-linked glycosylation patterns.

Bakker et al. (2001) (Proc. Natl. Acad. Sci. USA 96, 4692-4697 also describes stable expression of human β(1,4) galactosyltransferase in tobacco cells and partially galactosylated N-glycans (30%) on a monoclonal antibody expressed in such cells.

Bakker et al. (2006) described a tobacco plant expressing a hybrid β(1,4) galactosyltransferase and demonstrated that a mAB purified from leaves of plants expressing the hybrid enzyme displayed a N-glycan profile that featured high levels of galactose, undetectable xylose and a trace of fucose.

WO2004/057002 describes bryophyte plants and bryophyte plant cells comprising dysfunctional fucT and XylT genes and an introduced glycosyltransferase gene, such as a mammalian galactosyltransferase.

Huether et al. (2005) (Plant Biology, 7, 292-299) describes glycoengeering of moss lacking plant-specific sugar residues. Described are transgenic strains of the moss Physcomitrella patens in which the α (1,3) fucosyltransferase and β(1,2) xylosyltransferase genes were knocked out by targeted insertion of the human β(1,4) galactosyltransferase coding sequence in both of the plant genes.

The prior art however did not describe higher plant cells or plants which have no functional α (1,3) fucosyltransferase and β(1,2) xylosyltransferase activity while at the same time comprising a functional β(1,4) galactosyltransferase expressed under control of a plant-expressible promoter. To the contrary, the closest prior art document WO2004/057002 indicated that in higher plants it was not thought possible to suppress the activities of β(1,2) xylosyltransferase and of α (1,3) fucosyltransferase and moreover stresses the important differences between mosses and higher plants on the biochemical level.

Finally, none of the prior art documents demonstrated that plant glycoproteins obtained from plant cells or plants wherein β(1,2) xylosyltransferase and α (1,3) fucosyltransferase was eliminated and wherein further a β(1,4) galactosyltransferase was provided would exhibit a N-glycan profile with a higher level of galactosylation of the glycoproteins than in glycoproteins obtained from plants which have a normal β(1,2) xylosyltransferase and of α (1,3) fucosyltransferase activity level. Such a result is unexpected and inherently unpredictable, as the glycosylation pathway in eukaryotic organisms, but specifically in plants appears to be highly regulated and wherein modulation of the level of one glycotransferase appears to interact with the activity of other glycotransferase. See e.g. Bakker et al. 2006 (supra) where expression of a hybrid galactosyltransferase not only resulted in a N-glycan profile featuring high levels of galactose but also reduced levels of xylose and fucose.

The current invention provides method and means to alter the N-glycosylation pattern of glycoproteins in higher plant cells and plants as will become apparent from the following description, examples, drawings and claims provided herein.

### SUMMARY OF THE INVENTION

It is one object of the invention to provide a method to produce glycoproteins with altered glycosylation profile in higher plant cells, said method comprising the steps of providing a plant cell wherein said plant cell has a reduced level of β(1,2) xylosyltransferase and α (1,3) fucosyltransferase activity, preferably no detectable β(1,2) xylosyltransferase and no detectable α (1,3) fucosyltransferase activity and a functional β(1,4) galactosyltransferase activity, such as a mammalian or human β(1,4) galactosyltransferase and cultivating said cell and isolating glycoproteins from said cell. The reduced level of β(1,2) xylosyltransferase and α (1,3) fucosyltransferase activity may be the result of a null mutation in the endogenous β(1,2) xylosyltransferase and a (1,3) fucosyltransferase encoding genes or may be achieved by transcriptional or post-transcriptional silencing of the expression of endogenous β(1,2) xylosyltransferase and a (1,3) fucosyltransferase encoding genes. The β(1,4) galactosyltransferase is preferably expressed from a chimeric gene comprising a plant-expressible promoter operably linked to a DNA region encoding said β(1,4) galactosyltransferase and a DNA region involved in transcription termination and polyadenylation. The DNA region encoding the β(1,4) galactosyltransferase may be a nucleotide sequence encoding the amino acid sequence of SEQ ID No 11 such as the nucleotide sequence of SEQ ID No 10 from nucleotide position 523 to nucleotide position 1719. The glycoprotein may be a glycoprotein foreign to the higher plant cell and may be expressed from a chimeric gene comprising a plant expressible promoter operably linked to a coding region encoding the glycoprotein. The glycoprotein may also be expressed using a viral RNA vector. Preferred glycoproteins are therapeutic proteins such as monoclonal antibodies.

It is another object of the invention to provide a glycoprotein obtained by the methods described herein. The glycoproteins provided are derived from a higher plant cell having a complex N-glycan profile devoid of β(1,2) xylosyl and α (1,3) fucosyl and further comprising terminally linked β(1,4) galactosyl residues. In the glycoproteins according to the invention, a β(1,4) galactosyl residue may have been transferred to at least 30%, preferably at least 40% of the terminally linked N-acetylglucosamine residues, particularly when assessing the total glycoproteins, and not only secreted proteins.

In yet another embodiment, the invention provides cells of a higher plant comprising a reduced level of β(1,2) xylosyltransferase and α (1,3) fucosyltransferase activity or no β(1,2) xylosyltransferase and α (1,3) fucosyltransferase activity and a functional β(1,4) galactosyltransferase activity. The cells of the higher plant may comprise a chimeric gene including a plant-expressible promoter operably linked to a DNA region encoding a β(1,4) galactosyltransferase such as a mammalian or human or a hybrid β(1,4) galactosyltransferase and a DNA region involved in transcription termination and polyadenylation. The reduced level of β(1,2) xylosyltransferase and α (1,3) fucosyltransferase activity in the plant cell may be the result of a null mutation in the endogenous β(1,2) xylosyltransferase and α (1,3) fucosyltransferase encoding gene or may be achieved by transcriptional or post-transcriptional silencing of the expression of endogenous β(1,2) xylosyltransferase and α (1,3) fucosyltransferase encoding gene.

In yet another embodiment a higher plant consisting essentially of the plant cells described is provided.

The invention also provides a method to modify the N-glycosylation pattern of glycoproteins in higher plant cells, comprising the step of generating a plant cell wherein the plant cell has a reduced level of β(1,2) xylosyltransferase and α (1,3) fucosyltransferase activity and a functional β(1,4) galactosyltransferase.

### BRIEF DESCRIPTION OF THE FIGURES

FIG. 1: Western blot with antibodies specific for β(1,2) xylosyltransferase (panel A) and for α (1,3)-fucose containing N-glycans. WT: A. thaliana WT; XX/fafa/fbfb: FucTA and FucTB double knock-out; XX/fafa/FBFB: FucTA knock-out; xx/FAFA/FBFB: XylT knock-out; xx/fafa/fbfb: triple knock-out plants; Marker: BioRad Protein Marker.

FIG. 2A: MALDI-TOF mass spectra of N-glycans of endogenous proteins from a triple knock-out A. thaliana plant (xx/fafa/fbfb) (upper panel) and a wild-type A. thaliana plant (lower panel). The peaks in the mass-spectra relate to different N-glycans present on the endogenous proteins. The abbreviations for the glycans indicated with each peak are explained in FIG. 2B.

FIG. 2B: Schematic representation of the different glycan structures found in the MALDI-TOF analysis represented in FIG. 2A and corresponding abbreviations.

FIG. 3: Alignment of the isolated huGalT amino acid sequence used by Bakker et al. (supra) and the huGalT amino acid sequence of the current application. The four mismatches are indicated: position 10: glycine-arginine; position 76: glutamic acid-aspargic acid; position 292: leucine-serine; position 337: arginine-threonine.

FIG. 4: Detection of β(1,4) galactosyl residues present in the N-glycans of endogenous proteins using Western blotting with RCA₁₂₀. Samples were loaded before and after β(1,4) galactosidase treatment. Merker: BioRad Protein Marker; WT+gal: N-glycans from endogenous proteins from WT A. thaliana plants after treatment with β(1,4) galactosidase; WT: N-glycans from endogenous proteins from WT A. thaliana plants before treatment with β(1,4) galactosidase; huGalT/3KO 1+gal: N-glycans from endogenous proteins from A. thaliana plants having triple knock-out transgenic for HuGalT (xx/fafa/fbfb/HuGalT+) line 1 after treatment with β(1,4) galactosidase; huGalT/3KO 1: N-glycans from endogenous proteins from A. thaliana plants having triple knock-out transgenic for HuGalT (xx/fafa/fbfb/HuGalT+) line 1 before treatment with β(1,4) galactosidase; huGalT/3KO 2+gal: N-glycans from endogenous proteins from A. thaliana plants having triple knock-out transgenic for HuGalT (xx/fafa/fbfb/HuGalT+) line 2 after treatment with β(1,4) galactosidase; huGalT/3KO 2: N-glycans from endogenous proteins from A. thaliana plants having triple knock-out transgenic for HuGalT (xx/fafa/fbfb/HuGalT+) line 2 before treatment with β(1,4) galactosidase; huGalT1+gal: N-glycans from endogenous proteins from wild-type A. thaliana plants transgenic for HuGalT (XX/FAFA/FBFB/HuGalT+) line 1 after treatment with β(1,4) galactosidase; huGalT1: N-glycans from endogenous proteins from wild-type A. thaliana plants transgenic for HuGalT (XX/FAFA/FBFB/HuGalT+) line 1 before treatment with β(1,4) galactosidase; huGalT2+gal: N-glycans from endogenous proteins from wild-type A. thaliana plants transgenic for HuGalT (XX/FAFA/FBFB/HuGalT+) line 2 after treatment with β(1,4) galactosidase; huGalT2: N-glycans from endogenous proteins from wild-type A. thaliana plants transgenic for HuGalT (XX/FAFA/FBFB/HuGalT+) line 2 before treatment with β(1,4) galactosidase; pos control+gal: antibody produced in CHO cells after treatment with β(1,4) galactosidase; pos control: antibody produced in CHO cells before treatment with β(1,4) galactosidase.

FIG. 5: MALDI-TOF mass spectra of N-glycans of endogenous proteins prepared from A. thaliana plants selected from segregating progeny population obtained by crossing a wt A. thaliana plant (XX/FaFa/FbFb/—) with a triple knock-out A. thaliana plant hemizygous for HuGalT chimeric gene (xx/fafa/fbfb/HuGalT−), compared to the parent plant hemizygous for HuGalT. Panel A: MALDI-TOF spectrum from progeny plants heterozygous for the triple knock-out mutation (Xx/Fafa/Fbfb/—) which is similar to the wt spectrum in FIG. 2A lower panel. Panel B. MALDI-TOF spectrum from progeny plants heterozygous for the triple knock-out mutation and hemizygous for HuGalT (Xx/Fafa/Fbfb/HuGalT−). Panel C. MALDI-TOF spectrum from parent plants homozygous for the triple knock-out mutation and hemizygous for HuGalT (xx/fafa/fbfb/HuGalT−). The peaks in the mass-spectra relate to different N-glycans present on the endogenous proteins. The abbreviations for the glycans indicated with each peak are explained in Table 2.

### DETAILED DESCRIPTION OF DIFFERENT EMBODIMENT OF THE INVENTION

The current invention is based on the observation that N-glycans from glycoproteins derived from higher plant cells containing a chimeric plant-expressible huGalT which contained no functional β(1,2) xylosyltransferase or core-α (1,3) fucosyltransferase comprised more β(1,4) galactosyl residues than N-glycans from glycoproteins derived from transgenic higher plant cells which have a functional β(1,2) xylosyltransferase and core-α (1,3) fucosyltransferase and further contain a chimeric plant-expressible huGalT.

In a first embodiment, the invention thus provides a method to produce glycoproteins with altered glycosylation profile in higher plant cells comprising the steps of providing a plant cell wherein said plant cell has a reduced level of β(1,2) xylosyltransferase and α (1,3) fucosyltransferase activity and a functional β(1,4) galactosyltransferase activity; followed by cultivating the obtained cell and isolating glycoproteins from said cell.

As used herein “a higher plant cell” is a cell of plant belonging to the Angiospermae or the Gymospermae, but excluding Algae and Bryophyta. Preferably, the higher plant cell is a cell of a plant belonging to the Brassicaceae or the Solanaceae, including Arabidopsis or Nicotiana spp.

The level of β(1,2) xylosyltransferase and α (1,3) fucosyltransferase activity can conveniently be reduced or eliminated by identifying plant cells having a null mutation in all of the genes encoding β(1,2) xylosyltransferase and in all of the genes encoding a (1,3) fucosyltransferase.

Genes encoding α (1,3) fucosyltransferase in plants are well known and include the following database entries identifying experimentally demonstrated and putative FucT cDNA and gene sequences, parts thereof or homologous sequences: NM 112815 (Arabidopsis thaliana), NM103858 (Arabidopsis thaliana), AJ 618932 (Physcomitrella patens) At1g49710 (Arabidopsis thaliana) and At3g19280 (Arabidopsis thaliana). DQ789145 (Lemna minor), AY557602 (Medicago truncatula) Y18529 (Vigna radiata) AP004457 (Oryza sativa), AJ891040 encoding protein CAI70373 (Populus alba×Populus tremula) AY082445 encoding protein AAL99371 (Medicago sativa) AJ582182 encoding protein CAE46649 (Triticum aestivum) AJ582181 encoding protein CAE46648 (Hordeum vulgare) (all sequences herein incorporated by reference).

Genes encoding β(1,2) xylosyltransferase in plants are well known and include the following database entries identifying experimentally demonstrated and putative XylT cDNA and gene sequences, parts thereof or homologous sequences: AJ627182, AJ627183 (Nicotiana tabacum cv. Xanthi), AM179855 (Solanum tuberosum), AM179856 (Vitis vinifera), AJ891042 (Populus alba×Populus tremula), AY302251 (Medicago sativa), AJ864704 (Saccharum officinarum), AM179857 (Zea mays), AM179853 (Hordeum vulgare), AM179854 (Sorghum bicolor), BD434535, AJ277603, AJ272121, AF272852, AX236965 (Arabidopsis thaliana), AJ621918 (Oryza sativa), AR359783, AR359782, AR123000, AR123001 (Soybean), AJ618933 (Physcomitrella patens) and At5g55500 (Arabidopsis thaliana) as well as the nucleotide sequences from Nicotiana species described in application PCT/EP2007/002322 (all sequences herein incorporated by reference).

Based on the available sequences, the skilled person can isolate genes encoding a (1,3) fucosyltransferase or genes encoding β(1,2) xylosyltransferase from plants other than the plants mentioned above. Homologous nucleotide sequence may be identified and isolated by hybridization under stringent conditions using as probes identified nucleotide sequences.

“Stringent hybridization conditions” as used herein means that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C., preferably twice for about 10 minutes. Other hybridization and wash conditions are well known and are exemplified in Sambrook et al, Molecular Cloning: A Laboratory Manual, Second Edition, Cold Spring Harbor, N.Y. (1989), particularly chapter 11.

Nucleotide sequences obtained in this way should be verified for encoding a polypeptide having an amino acid sequence which is at least 80% to 95% identical to a known α (1,3) fucosyltransferase or β(1,2) xylosyltransferase from plants.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch 1970) The computer-assisted sequence alignment above, can be conveniently performed using standard software program such as GAP which is part of the Wisconsin Package Version 10.1 (Genetics Computer Group, Madison, Wis., USA) using the default scoring matrix with a gap creation penalty of 50 and a gap extension penalty of 3. Sequences are indicated as “essentially similar” when such sequence have a sequence identity of at least about 75%, particularly at least about 80%, more particularly at least about 85%, quite particularly about 90%, especially about 95%, more especially about 100%, quite especially are identical. It is clear than when RNA sequences are the to be essentially similar or have a certain degree of sequence identity with DNA sequences, thymine (T) in the DNA sequence is considered equal to uracil (U) in the RNA sequence.

Other sequences encoding α (1,3) fucosyltransferase or β(1,2) xylosyltransferase may also be obtained by DNA amplification using oligonucleotides specific for genes encoding α (1,3) fucosyltransferase or β(1,2) xylosyltransferase as primers, such as but not limited to oligonucleotides comprising about 20 to about 50 consecutive nucleotides from the known nucleotide sequences or their complement.

The art also provides for numerous methods to isolate and identify plant cells having a mutation in a particular gene.

Mutants having a deletion or other lesion in the α (1,3) fucosyltransferase or β(1,2) xylosyltransferase encoding genes can conveniently be recognized using e.g. a method named “Targeting induced local lesions in genomes (TILLING)”. Plant Physiol. 2000 June; 123(2):439-42. Plant cells having a mutation in the desired gene may also be identified in other ways, e.g. through amplification and nucleotide sequence determination of the gene of interest. Null mutations may include e.g. genes with insertions in the coding region or gene with premature stop codons or mutations which interfere with the correct splicing. Mutants may be induced by treatment with ionizing radiation or by treatment with chemical mutagens such as EMS.

The level of β(1,2) xylosyltransferase and α (1,3) fucosyltransferase activity can also be conveniently be reduced or eliminated by transcriptional or post-transcriptional silencing of the expression of endogenous β(1,2) xylosyltransferase and α (1,3) fucosyltransferase encoding genes. To this end, a silencing RNA molecule is introduced in the plant cells targeting the endogenous β(1,2) xylosyltransferase and α (1,3) fucosyltransferase encoding genes. As used herein, “silencing RNA” or “silencing RNA molecule” refers to any RNA molecule, which upon introduction into a plant cell, reduces the expression of a target gene. Such silencing RNA may e.g. be so-called “antisense RNA”, whereby the RNA molecule comprises a sequence of at least 20 consecutive nucleotides having 95% sequence identity to the complement of the sequence of the target nucleic acid, preferably the coding sequence of the target gene. However, antisense RNA may also be directed to regulatory sequences of target genes, including the promoter sequences and transcription termination and polyadenylation signals. Silencing RNA further includes so-called “sense RNA” whereby the RNA molecule comprises a sequence of at least 20 consecutive nucleotides having 95% sequence identity to the sequence of the target nucleic acid. Other silencing RNA may be “unpolyadenylated RNA” comprising at least 20 consecutive nucleotides having 95% sequence identity to the complement of the sequence of the target nucleic acid, such as described in WO01/12824 or U.S. Pat. No. 6,423,885 (both documents herein incorporated by reference). Yet another type of silencing RNA is an RNA molecule as described in WO03/076619 (herein incorporated by reference) comprising at least 20 consecutive nucleotides having 95% sequence identity to the sequence of the target nucleic acid or the complement thereof, and further comprising a largely-double stranded region as described in WO03/076619 (including largely double stranded regions comprising a nuclear localization signal from a viroid of the Potato spindle tuber viroid-type or comprising CUG trinucleotide repeats). Silencing RNA may also be double stranded RNA comprising a sense and antisense strand as herein defined, wherein the sense and antisense strand are capable of base-pairing with each other to form a double stranded RNA region (preferably the said at least 20 consecutive nucleotides of the sense and antisense RNA are complementary to each other). The sense and antisense region may also be present within one RNA molecule such that a hairpin RNA (hpRNA) can be formed when the sense and antisense region form a double stranded RNA region. hpRNA is well-known within the art (see e.g. WO99/53050; herein incorporated by reference). The hpRNA may be classified as long hpRNA, having long, sense and antisense regions which can be largely complementary, but need not be entirely complementary (typically larger than about 200 bp, ranging between 200-1000 bp). hpRNA can also be rather small ranging in size from about 30 to about 42 bp, but not much longer than 94 by (see WO04/073390, herein incorporated by reference). Silencing RNA may also be artificial micro-RNA molecules as described e.g. in WO2005/052170, WO2005/047505 or US 2005/0144667 (all documents incorporated herein by reference)

In another embodiment, the silencing RNA molecules are provided to the plant cell or plant by producing a transgenic plant cell or plant comprising a chimeric gene capable of producing a silencing RNA molecule, particularly a double stranded RNA (“dsRNA”) molecule, wherein the complementary RNA strands of such a dsRNA molecule comprises a part of a nucleotide sequence encoding a XylT or FucT protein.

The plant cells according to the invention also need to comprise a β(1,4) galactosyltransferase activity. Conveniently, such activity may be introduced into plant cells by providing them with a chimeric gene comprising a plant-expressible promoter operably linked to a DNA region encoding a β(1,4) galactosyltransferase and optionally a 3′ end region involving in transcription termination and polyadenylation functional in plant cells.

As used herein, the term “plant-expressible promoter” means a DNA sequence that is capable of controlling (initiating) transcription in a plant cell. This includes any promoter of plant origin, but also any promoter of non-plant origin which is capable of directing transcription in a plant cell, i.e., certain promoters of viral or bacterial origin such as the CaMV35S (Hapster et al., 1988), the subterranean clover virus promoter No 4 or No 7 (WO9606932), or T-DNA gene promoters but also tissue-specific or organ-specific promoters including but not limited to seed-specific promoters (e.g., WO89/03887), organ-primordia specific promoters (An et al., 1996), stem-specific promoters (Keller et al., 1988), leaf specific promoters (Hudspeth et al., 1989), mesophyl-specific promoters (such as the light-inducible Rubisco promoters), root-specific promoters (Keller et al., 1989), tuber-specific promoters (Keil et al., 1989), vascular tissue specific promoters (Peleman et al., 1989), stamen-selective promoters (WO 89/10396, WO 92/13956), dehiscence zone specific promoters (WO 97/13865) and the like.

Regions encoding a β(1,4) galactosyltransferase are preferably obtained from mammalian organisms, including humans, but may be obtained from other organisms as well. NM022305 (Mus musculus) NM146045 (Mus musculus) NM 004776 (Homo sapiens) NM 001497 (Homo sapiens) are a few database entries for genes encoding a β(1,4) galactosyltransferase. Others database entries for β(1,4) galactosyltransferases include AAB05218 (Gallus gallus), XP693272 (Danio rerio), CAF95423 (Tetraodon nigroviridis) or NP001016664 (Xenopus tropicalis) (all sequences herein incorporated by reference). The β(1,4) galactosyltransferase may be a hybrid β(1,4) galactosyltransferase i.e. a galactosyltransferase comprising a transmembrane region from another glycosyltransferase as described by e.g. by WO03/078637.

According to the invention, the N-glycan profile of glycoproteins may be altered or modified. The glycoproteins may be glycoproteins endogeneous to the cell of the higher plant, and may result in altered functionality, folding or half-life of these proteins. Glycoproteins also include proteins which are foreign to the cell of the higher plant, i.e. which are not normally expressed in such plant cells in nature. These may include mammalian or human proteins, which can be used as therapeutics such as e.g. monoclonal antibodies. Conveniently, the foreign glycoproteins may be expressed from chimeric genes comprising a plant-expressible promoter and the coding region of the glycoprotein of interest, whereby the chimeric gene is stably integrated in the genome of the plant cell. Methods to express foreign proteins in plant cells are well known in the art. Alternatively, the foreign glycoproteins may also be expressed in a transient manner, e.g. using the viral vectors and methods described in WO02/088369, WO2006/079546 and WO2006/012906 or using the viral vectors described in WO89/08145, WO93/03161 and WO96/40867 or WO96/12028. The methods of the invention lead to the presence of a higher proportion of glycoproteins with a human glycosylation profile, such as complex biantennary glycosylated proteins having galactosyl residues with a β(1,4) linkage to both (pre)terminal GlcNac residues, and without fucosyl residue linked α1,3 to the core structure or β1,2 linked xylosyl residues (AA in abbreviated glycan structure nomenclature—see Table 2).

The methods and means described herein are believed to be suitable for all plant cells and plants, gymnosperms and angiosperms, both dicotyledonous and monocotyledonous plant cells and plants including but not limited to Arabidopsis, alfalfa, barley, bean, corn or maize, cotton, flax, oat, pea, rape, rice, rye, safflower, sorghum, soybean, sunflower, tobacco and other Nicotiana species, including Nicotiana benthamiana, wheat, asparagus, beet, broccoli, cabbage, carrot, cauliflower, celery, cucumber, eggplant, lettuce, onion, oilseed rape, pepper, potato, pumpkin, radish, spinach, squash, tomato, zucchini, almond, apple, apricot, banana, blackberry, blueberry, cacao, cherry, coconut, cranberry, date, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut and watermelon Brassica vegetables, sugarcane, vegetables (including chicory, lettuce, tomato) and sugarbeet.

Methods for the introduction of chimeric genes into plants are well known in the art and include Agrobacterium-mediated transformation, particle gun delivery, microinjection, electroporation of intact cells, polyethyleneglycol-mediated protoplast transformation, electroporation of protoplasts, liposome-mediated transformation, silicon-whiskers mediated transformation etc. The transformed cells obtained in this way may then be regenerated into mature fertile plants.

Gametes, seeds, embryos, progeny, hybrids of plants, or plant tissues including stems, leaves, stamen, ovaria, roots, meristems, flowers, seeds, fruits, fibers comprising the chimeric genes of the present invention, which are produced by traditional breeding methods are also included within the scope of the present invention.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region which is functionally or structurally defined, may comprise additional DNA regions etc.

The following non-limiting Examples describe the identification of XylT−, FucT−, GalT+ plant cells and analysis of the glycoproteins contained therein.

Unless stated otherwise in the Examples, all recombinant techniques are carried out according to standard protocols as described in “Sambrook J and Russell D W (eds.) (2001) Molecular Cloning: A Laboratory Manual, 3rd Edition, Cold Spring Harbor Laboratory Press, New York” and in “Ausubel F A, Brent R, Kingston R E, Moore D D, Seidman J G, Smith J A and Struhl K (eds.) (2006) Current Protocols in Molecular Biology. John Wiley & Sons, New York”. Standard materials and references are described in “Croy R D D (ed.) (1993) Plant Molecular Biology LabFax, BIOS Scientific Publishers Ltd., Oxford and Blackwell Scientific Publications, Oxford” and in “Brown T A, (1998) Molecular Biology LabFax, 2nd Edition, Academic Press, San Diego”. Standard materials and methods for polymerase chain reactions (PCR) can be found in “McPherson M J and Møller S G (2000) PCR (The Basics), BIOS Scientific Publishers Ltd., Oxford” and in “PCR Applications Manual, 3rd Edition (2006), Roche Diagnostics GmbH, Mannheim or www.roche-applied-science.com”

Throughout the description and Examples, reference is made to the following sequences:
SEQ ID No 1: Nucleotide sequence of oligonucleotide used as FucTA Left primer (LP)
SEQ ID No 2: Nucleotide sequence of oligonucleotide used as FucTA Right primer (RP)
SEQ ID No 3: Nucleotide sequence of oligonucleotide used as FucTB Left primer (LP)
SEQ ID No 4: Nucleotide sequence of oligonucleotide used as FucTB Right primer (LP)
SEQ ID No 5: Nucleotide sequence of oligonucleotide used as XylT Left primer (LP)
SEQ ID No 6: Nucleotide sequence of oligonucleotide used as XylT Right primer (LP)
SEQ ID No 7: Nucleotide sequence of oligonucleotide used as Left Border T-DNA primer (LB)
SEQ ID No 8: Nucleotide sequence of oligonucleotide used as Forward huGalT primer
SEQ ID No 9: Nucleotide sequence of oligonucleotide used as Reverse huGalT primer
SEQ ID No 10: Nucleotide sequence of plant-expressible huGalT chimeric gene
SEQ ID No 11: Amino acid sequence of huGalT
SEQ ID No 12: A. thaliana XylT gene At5g55500
SEQ ID No 13: A. thaliana FucTB gene At1g49710
SEQ ID No 14: A. thaliana FucTA gene At3g19280

### EXAMPLES

### Example 1

### Identification of a Triple Knock-Out Arabidopsis Thaliana with Homozygous T-DNA Insertions in FucTA, FucTB and XylT Genes

A. thaliana lines containing a T-DNA insertion in either the XylT gene (At5g55500), the FucTA gene (At3g19280) and the FucTB gene (At1g49710) are available in the public A. thaliana T-DNA insertion database SIgnAL (http://signal.salk.edu) as Salk_{—}42226, Salk_{—}87481 and Salk_{—}63355 respectively.

Plant lines carrying a homozygous T-DNA insertion, plant lines carrying a heterozygous T-DNA insertion and plant lines carrying no T-DNA insertion at the desired locus can be discriminated using two PCR reactions for each insertion. The first PCR reaction is determinative for the presence of a T-DNA insertion in the desired locus whereby respectively a primer recognizing a target in the inserted T-DNA (LB; SEQ ID No 7) and a primer recognizing the target flanking the T-DNA specific for the locus (such as FucTA RP of SEQ ID No 2; FucTB RP of SEQ ID No 4 or XylT RP of SEQ ID No 6) are used and PCR fragment of about 400 by is amplified. The second PCR reaction is determinative for the absence of a T-DNA insertion in the desired locus whereby respectively a primer recognizing a target left of the T-DNA insertion (such as FucTA LP of SEQ ID No 1; FucTB LP of SEQ ID No 3 or XylT RP of SEQ ID No 5) and a primer recognizing the target flanking the T-DNA specific for the locus at the other side (such as FucTA RP of SEQ ID No 2; FucTB RP of SEQ ID No 4 or XylT RP of SEQ ID No 6) are used and a PCR fragment of about 900 by is amplified.

Plant lines lacking a T-DNA insertion will react positively in the PCR reaction using the specific LP and RP primers and a fragment of about 900 by will be amplified. Such plants will also react negatively in the PCR reaction using the LB and specific RP primers.

Plant lines homozygous for the T-DNA insertion will react positively in the PCR reaction using the LB and specific RP primers and a fragment of about 400 by will be amplified. Such plants will react negatively in the PCR reaction using the specific LP/RP primer combination.

Plant lines heterozygous for the T-DNA insertion will react positively in the PCR reaction using the LB and specific RP primers and a fragment of about 400 by will be amplified. Such plants will also react positively in the PCR reaction using the specific LP/RP primer combination and a fragment of about 900 by will be amplified.

With a first cross between homozygous FucTA knock-out plants (fafa/FBFB) and FucTB knock-out plants (FAFA/fbfb) a heterozygous FucTA/FucTB knock-out line (FAfa/FBfb) could be identified using the PCR reactions described above.

These plants (XX/FAfa/FBfb) were crossed with a homozygous XylT knock-out line (xx/FAFA/FBFB) and progeny of the plants were screened using the above mentioned PCR reactions for the heterozygosity in all three loci (xX/faFA/fbFB). Such triple knock-out plants were selfed and progeny was screened for homozygosity for the T-DNA insertion in all three loci using the above described PCR reactions.

2 Lines out of 300 were identified as positive in the LB/specific RP primer PCR reaction and negative in the specific LP/specific RP primer PCR reaction. The lines are thus potential candidate triple knock-out homozygous plants.

### Example 2

### Western Blot and MALDI-TOF Mass Spectrometry Analysis of N-Glycans of Endogenous Glycoproteins of the Triple Knock-Out A. Thaliana Lines.

Proteins were extracted from the triple knock-out homozygous A. thaliana plants described in Example 1, separated on polyacrylamide gel and blotted to a PVDF membrane. The resulting blots were treated in a Western Blot using anti-horse radish peroxidase polyclonal antibodies which had been separated in a fraction recognizing β(1,2) xylosyl residues and a fraction recognizing core-α (1,3) fucosyl residues through affinity chromatography using insect phospholipase bound sepharose.

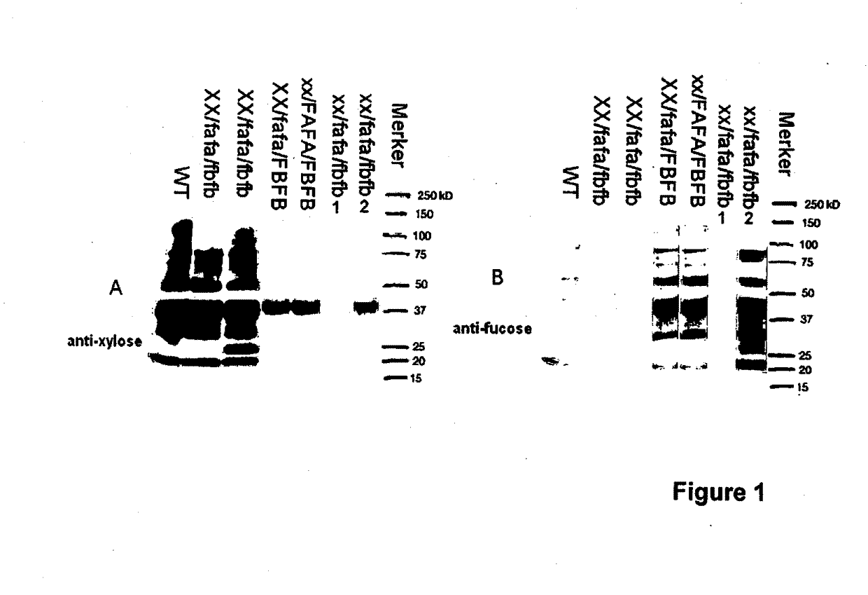

The result is displayed in FIG. 1. In wild type plants, XylT, FucTA, FucTB and FucTA/FucTB knock plants an intense coloration of different bands could be observed. In one of the lines of the triple knock-out plants, no reaction with the anti HRP fraction specific for core-α (1,3) fucosyl residues could be observed while only a faint band was observed after reaction with the anti-HRP fraction specific for β(1,2) xylosyl residues. The latter may be explained as the presence of an endogenous peroxidase recognized by the antiHRP polyclonal antibody.

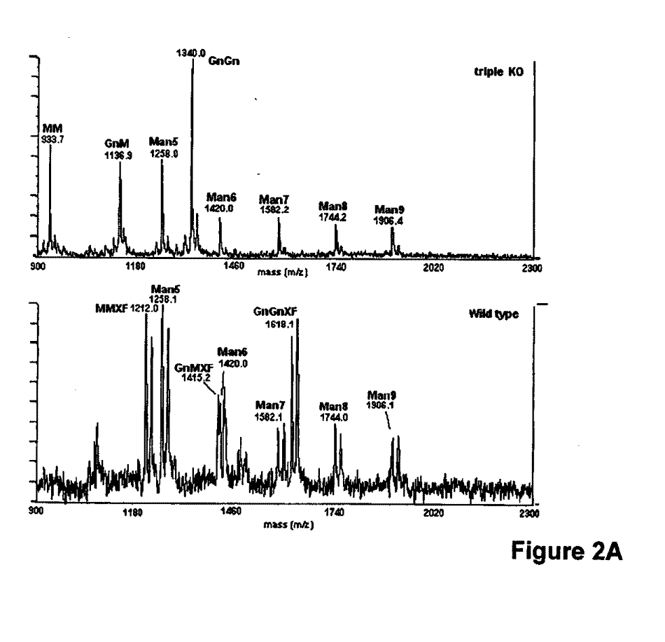

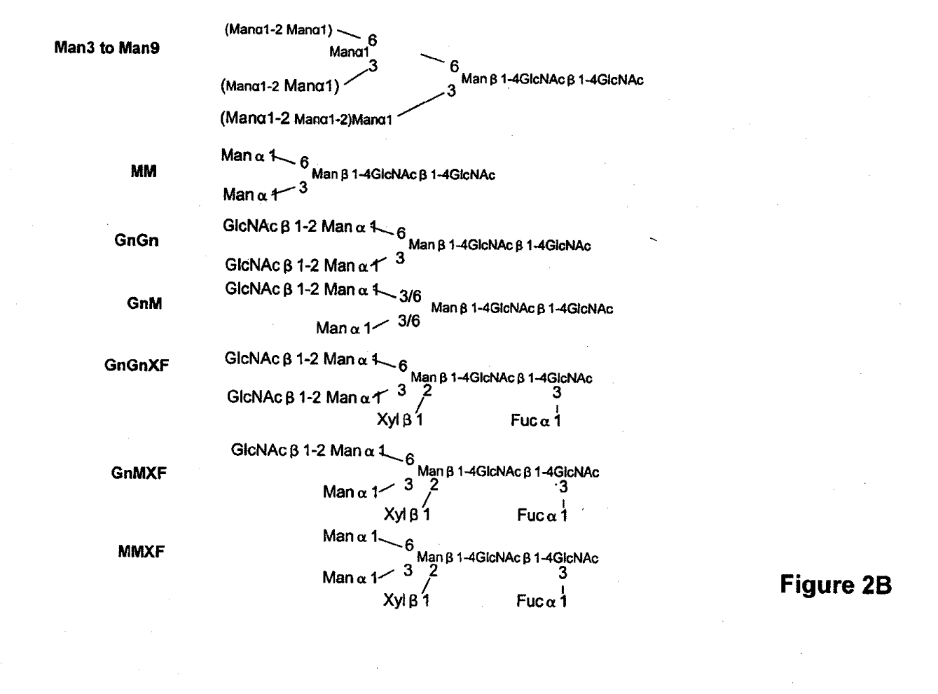

Proteins were extracted from the plant line reacting negatively both for the presence of xylose and fucose residues, treated with pepsine, and peptide-N-glycosidase A to obtain N-glycans which were subjected to MALDI-TOF analysis. The result of this analysis is displayed in FIG. 2A. Whereas the massaspectra obtained from N-glycans of a WT plants clearly contain a number of peaks distinctive for the presence of xylose and fucose residues (MMXF, GnMXF, GnGnXF; for explanation of the abbreviations see FIG. 2B) no such peaks were observed in the N-glycans obtained from the triple knock-out plants described in Example 1.

Table 1 represents the relative proportion of the different N-glycans present in the endogenous glycoproteins calculated on the basis of the surface of the different peaks in the MALDI-TOF mass spectra. In wild type plants 32% of the N-glycans present on the endogenous proteins contained one or two terminal GlcNac residues, whereas in the triple knock plants this percentage was 44%.

TABLE 1
Relative proportion of the N-glycans

| | N-glycan | |
|---|---|---|
| Mass | Triple knockout | Percentage |
| 933.8 | MM | 12 |
| 1137.0 | GnM | 11 |
| 1258.1 | Man5 | 14 |
| 1340.2 | GnGn | 33 |
| 1420.3 | Man6 | 7 |
| 1582.4 | Man7 | 8 |
| 1744.5 | Man8 | 8 |
| 1906.7 | Man9 | 8 |
| | N-glycan | |
| Mass | Wild type | Percentage |
| 1212.1 | MMXF | 15 |
| 1258.1 | Man5 | 17 |
| 1415.0 | GnMXF | 10 |
| 1420.3 | Man6 | 9 |
| 1582.4 | Man7 | 8 |
| 1618.3 | GnGnXF | 22 |
| 1744.5 | Man8 | 10 |
| 1906.7 | Man9 | 9 |

### Example 3

### Construction of a Chimeric Gene for Expression of Human GalT in Plants

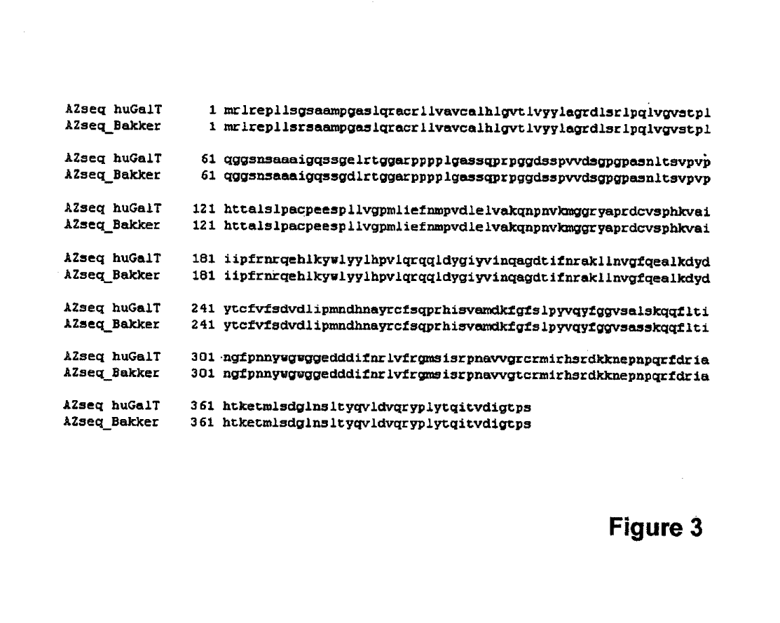

Isolation of huGalT coding sequence was done by PCR reaction using oligonucleotide primers having the nucleotide sequence of SEQ ID No 8 and SEQ ID No 9. The nucleotide sequence was determined (SEQ ID No 10 from nucleotide position 523 to nucleotide position 1719) and the encoded amino acid sequence was compared to that of the huGalT used by Bakker et al. 2001 (supra) (FIG. 3). Four variations were found on positions 10, 76, 292 and 337.

Using standard recombinant techniques a chimeric gene was constructed containing the following operably linked DNA regions:
a CaMV 35S promoter (SEQ ID No 10 from nucleotide 1 to nucleotide 452)
an untranslated leader sequence Cab22L (SEQ ID No 10 from nucleotide 453 to nucleotide 516)
a huGalT encoding DNA region (SEQ ID No 10 from nucleotide position 523 to nucleotide position 1719)
a 3′ end of the CaMV 35S transcript (SEQ ID No 10 from nucleotide position 1725 to nucleotide position 1949).

The chimeric DNA encoding huGalT was introduced into a T-DNA vector further comprising a glyphosate-resistance gene and introduced into Agrobacterium tumefaciens comprising a helper Ti-plasmide.

### Example 4

### Isolation of Transgenic A. Thaliana Lines Comprising Human GalT

Using the floral dip method, transgenic A. thaliana plants comprising a plant-expressible huGalT chimeric gene were isolated by spraying the population of potential transgenic plants first with glyphosate, followed by a further identification using PCR reaction with primers specific for the huGalT chimeric gene.

The chimeric gene was introduced both into WT A. thaliana plants as well as into the triple knock-out A. thaliana lines described in Example 1.

### Example 5

### Analysis of N-Glycans of Endogenous Glycoproteins in the Triple Knock-Out FucTA−, FucTB−, XyIT−, GalT+ Lines

Proteins were isolated from leaf material of plants comprising the huGalT chimeric gene as described in Example 4 and analysed by Western blotting using HRP-conjugated RCA₁₂₀. RCA₁₂₀ is a lectin from Ricinus communis which binds both β(1,4) and β(1,3) bound galactosyl residues. Since plants also contain β(1,3) bound galactosyl, protein samples treated with a β(1,4) galactosidase were also included in the Western Blot analysis.

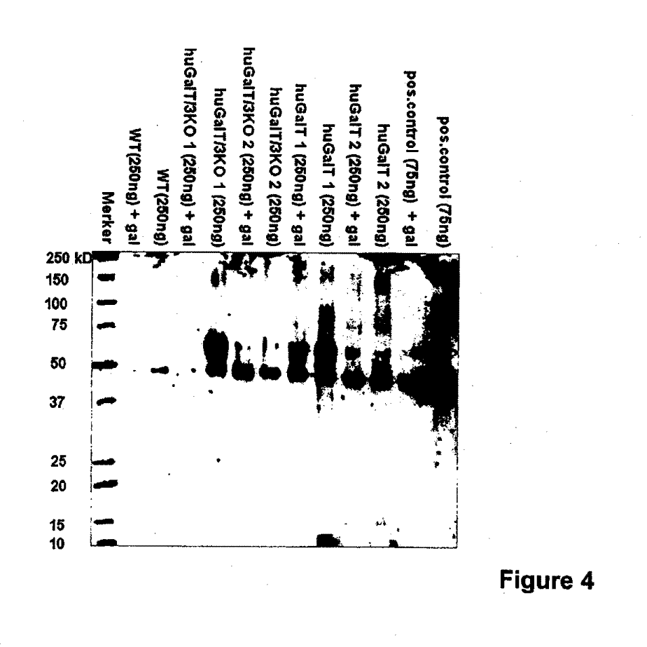

FIG. 4 represents the results obtained from the above described Western Blotting. Both the lanes of proteins obtained from transgenic wt plants line 1 comprising the huGalT and from the triple knock-out plants line 1 comprising the huGalT exhibited a significant signal in the lane prior to β(1,4) galactosidase treatment, which was significantly decreased after β(1,4) galactosidase treatment indicating that the glycol-proteins contained a significant amount of β(1,4) bound galactosidyl residues. However, the decrease in the signal after β(1,4) galactosidase treatment was more pronounced in the transgenic huGalT containing triple knock out plants than in the transgenic huGalT containing WT plants, indicating a higher presence of β(1,4) galactosidase in the N-glycan of the endogenous proteins of the former plants.

### Example 6

### Generation of Isogenic Homozygous Triple Knock-Out Arabidopsis Thaliana Plants with Homozygous T-DNA Insertions in FucTA, FucTB and XylT Genes and Hemizygous for the Presence of HuGalT and Hemizygous Triple Knock-Out Arabidopsis Thaliana Plants with Hemizygous T-DNA Insertions in FucTA, FucTB and XylT Genes Hemizygous for the Presence of HuGalT

The wt and triple knock-out transgenic plants generated in Example 4 originate from independent transformation events, with potentially a different expression pattern of the HuGalT transgene, thereby complicating the comparison of the N-glycan analysis of endogenous glycoproteins isolated from both types of plants.

To generate isogenic transgenic HuGalT lines which differ only in the zygosity status for the T-DNA insertions in xylosyltransferase and fucosyltransferase genes, a β(1,2) xylosyltransferase and α (1,3) fucosyltransferase deficient A. thaliana line as identified in Example 1 (homozygous triple knock out mutant) was transformed with a chimeric HuGalT gene as described in Example 3 using the floral dip method.

Glyphosate tolerant progeny plants were verified for the intact presence of the HuGalT chimeric gene using PCR amplification specific for HuGalT and CaMV35S promoter. The candidate transformants were also verified for homozygous triple knock-out status as described in Example 1. By Southern blot analysis, candidate transformed plants which had a single insertion of the HuGalT comprising T-DNA were selected. By Western blotting using HRP-conjugated RCA₁₂₀ as described in Example 5, candidate transformed plants with a good expression level of HuGalT, as judged from the difference in RCA120 binding before and after treatment with β(1,4) galactosidase, were selected.

Transformed A. thaliana plants, homozygous for the presence of T-DNA insertions in the xylosyl and fucosyltransferase genes, comprising a single chimeric HuGalT gene with good expression in hemizygous state (xx/fafa/fbfb/HuGalT−) were crossed with isogenic wild type A. thaliana plants (XX/FaFa/FbFfb/—). Progeny plants may have the following genotypes:
a. Xx/Fafa/Fbfb/HuGalT−
b. Xx/Fafa/Fbfb/—

The zygosity status for either the chimeric gene or the T-DNA insertions in the xylosyl and fucosyltransferase genes was checked as described above. The HuGalT chimeric gene in the triple knock-out parent line and the transgenic hemizygous progeny plants (genotype a above) are integrated at the same locus in the genome and thus allow a comparison of the N-glycan profiles of the glycoproteins of plants differing in xylosyl/fucosyl transferase activity without potentially distortion caused by the “position effect” of the HuGalT gene. Analysis of the N-glycan profile of the plant with a genotype as described in b above, allows to verify that plants with a hemizygous knock-out in xylosyl/fucosyltransferase genes behave as a wt plant with regard to the N-glycan profile of endoglycoproteins.

### Example 7

### Analysis of N-glycans of Endogenous Glycoproteins of the Different Plants Described in Example 6

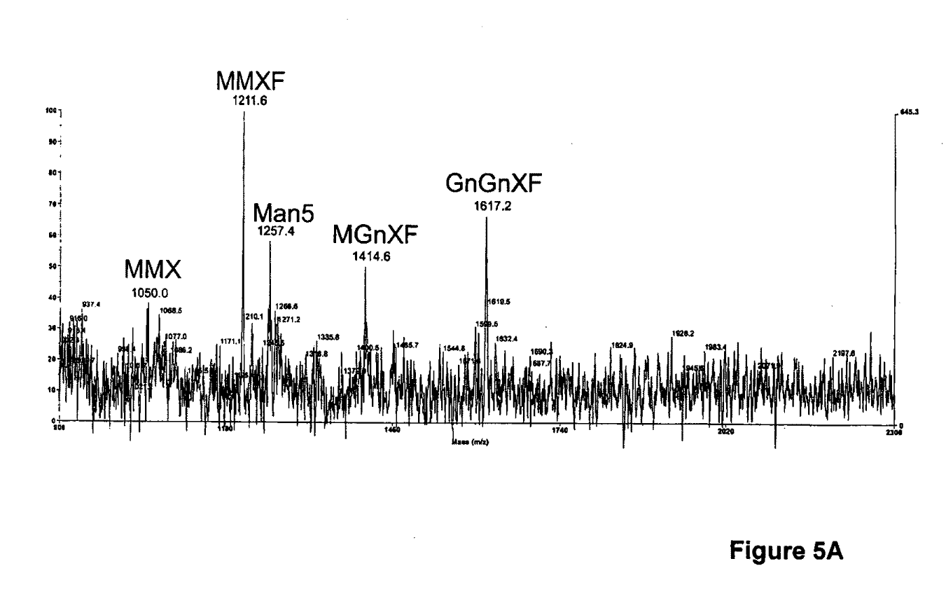

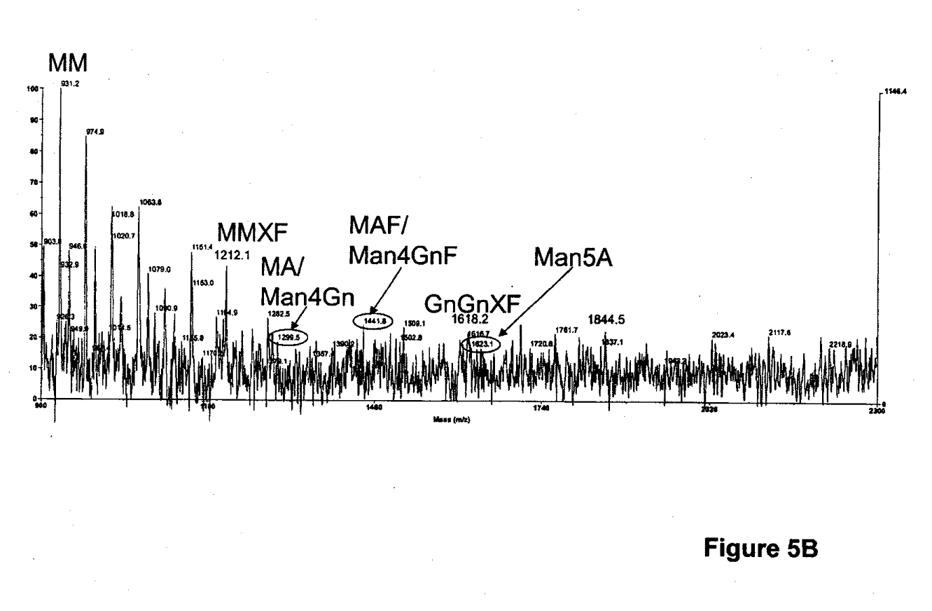

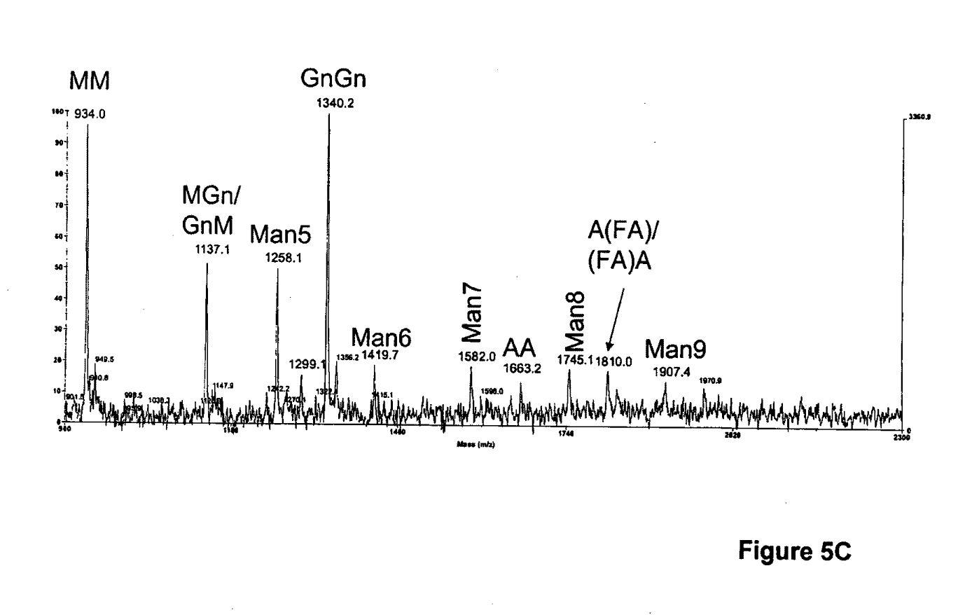

Endoglycoproteins were prepared as described in Example 1 from
a. Progeny plants from Example 6 without HuGalT with genotype Xx/Fafa/Fbfb/—
b. Progeny plants from Example 6 with HuGalT with genotype Xx/Fafa/Fbfb/HuGalT−
c. Parent plants from Example 6 with HuGalT with genotype xx/fafa/fbfb/HuGalT−
   and subjected to MALDI-TOF analysis. The mass spectra are represented in FIGS. 5A-C, respectively. The different glycan structures are indicated by their abbreviated nomenclature (explained in following table 2).

The following conclusions can be drawn from the analysis:
1. Plants hemizygous for the T-DNA insertions in the xylosyl and fucosyltransferase genes have a N-glycan profile similar to the wild-type plants (compare FIG. 5A with FIG. 2B—particularly peaks indicated by MMX, MMXF, MGnXF and GnGbXF).
2. The N-glycan profile of plants hemizygous for T-DNA insertions in the xylosyl and fucosyltransferase genes wherein a HuGalT chimeric gene is expressed demonstrates that galactosylation is taking place, however the structures are complex and of an undesired type (Man5A, MAF) or less desired type (MA) (FIG. 5B)
3. Only plants with a homozygous triple knockout of the xylosyltransferase and fucosyltransferase type and further expressing a HuGalT gene, the N-glycans contain the desired human-type glycosylation pattern indicated by the peak denominated AA (FIG. 5C). Further present in the profile are structures indicated by A(FA) or (FA)A which although in se undesired, are nevertheless complex galactosylated bi-antennary glycans, which however appear to have undergone a further fucosylation through the activity of an α 1,4-fucosyltransferase.

TABLE 2
Abbreviated nomenclature of different glycan structures

| | |
|---|---|
| GnGn | 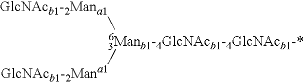 |
| GnM | 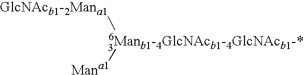 |
| MGn | 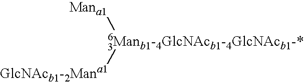 |
| GnGnXF | 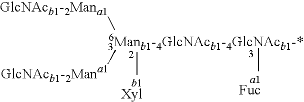 |
| MGnXF | 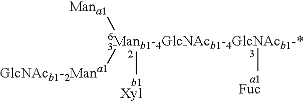 |
| Man4Gn | 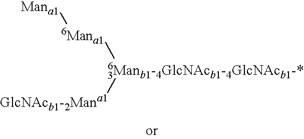 |
| | 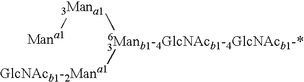 |
| Man4GnF | 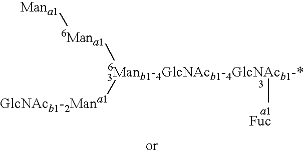 |
| | 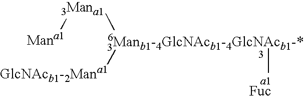 |
| Man5 | 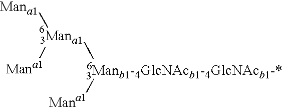 |
| Man5A | 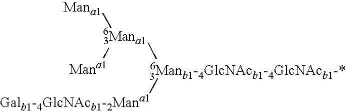 |
| Man6 | 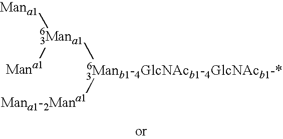 |
| | 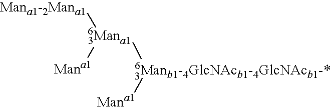 |
| Man7 | 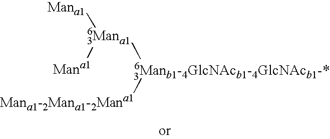 |
| | 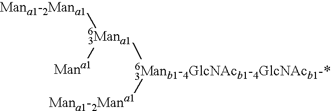 |
| Man8 | 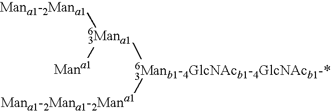 |
| Man9 | 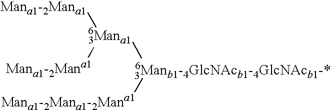 |
| MM | 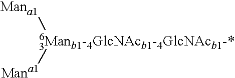 |
| MMX | 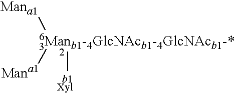 |
| MMXF | 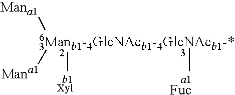 |
| MA | 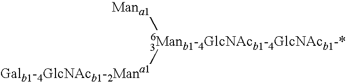 |
| MAF | 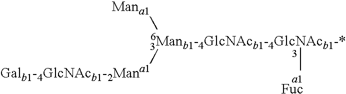 |
| AA | 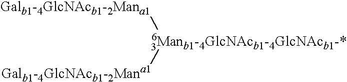 |
| A(FA) | 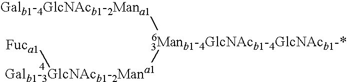 |
| (FA)A | 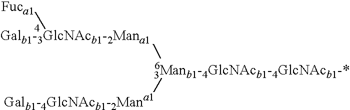 |

### Example 8

### Construction of a Chimeric Gene for Expression of a Foreign Glycoprotein in Plant Cells, Isolation of Transgenic A. Thaliana Lines Expressing a Foreign Glycoprotein and Isolating A. Thaliana FucTA−, FucTB−, XylT−, GalT+ Lines Expressing a Foreign Glycoprotein.

A foreign glycoprotein encoding chimeric gene is generated using standard recombinant DNA techniques. The chimeric gene is introduced into a T-DNA vector further comprising a chimeric gene encoding a selectable marker protein, such a chimeric phosphinotricin resistance marker gene and transgenic A. thaliana plants comprising such T-DNAs are generated.

The transgenic plant expressing the foreign glycoprotein are crossed with a XylT⁻, FucTA⁻, FucTB⁻ plant expressing huGalT as described in Example 4 or 5 and progeny plants are selected by spraying with glufosinate and glyphosate. The surviving plants are screened by PCR for the presence of both transgenes. Glycoproteins isolated from the identified progeny plants have no β(1,2) xylosyl- or α (1,3) fucosyl-residues and exhibit a high amount of β(1,4) galactosyl residues in their N-linked glycan structures. The glycan structures exhibit the desired AA-type glycans.

1. A method to produce glycoproteins with an altered glycosylation profile in higher plant cells, said method comprising the steps of:
a. providing a plant cell comprising a reduced level of β(1,2) xylosyltransferase and α(1,3) fucosyltransferase activity, and a functional β(1,4) galactosyltransferase activity; and
b. cultivating said plant cell and isolating glycoproteins from said plant cell.

2. The method according to claim 1, wherein said plant cell has no detectable β(1,2) xylosyltransferase and no detectable α(1,3) fucosyltransferase activity.

3. The method according to claim 1, wherein said β(1,4) galactosyltransferase activity is encoded by a mammalian β(1,4) galactosyltransferase.

4. The method according to claim 3, wherein said mammalian β(1,4) galactosyltransferase is a human β(1,4) galactosyltransferase.

5. The method according to claim 1, wherein said β(1,4) galactosyltransferase activity is encoded by a hybrid β(1,4) galactosyltransferase.

6. The method according to claim 1, wherein said reduced level of β(1,2) xylosyltransferase and α (1,3) fucosyltransferase activity is the result of a null mutation in the endogenous β(1,2) xylosyltransferase and α (1,3) fucosyltransferase encoding genes.

7. The method according to claim 1, wherein said reduced level of β(1,2) xylosyltransferase and α (1,3) fucosyltransferase activity is achieved by transcriptional or post-transcriptional silencing of the expression of endogenous β(1,2) xylosyltransferase and α (1,3) fucosyltransferase encoding genes.

8. The method according to claim 1, wherein said β(1,4) galactosyltransferase is expressed from a chimeric gene comprising the following operably linked nucleic acid molecules:
i. a plant-expressible promoter
ii. a DNA region encoding said β(1,4) galactosyltransferase; and
iii a DNA region involved in transcription termination and polyadenylation.

9. The method according to claim 8, wherein said DNA region encoding said β(1,4) galactosyltransferase comprise a nucleotide sequence encoding the amino acid sequence of SEQ ID No 11.

10. The method according to claim 1, wherein said glycoprotein is a glycoprotein foreign to said higher plant cell.

11. The method according to claim 1, wherein said glycoprotein is expressed from a chimeric gene comprising a plant expressible promoter operably linked to a coding region encoding said glycoprotein.

12. The method according to claim 1, wherein said glycoprotein is expressed using a viral RNA vector.

13. The method according to claim 1, wherein said glycoprotein is a mammalian protein.

14. The method according to claim 1, wherein said glycoprotein is a therapeutic protein.

15. The method according to claim 1, wherein said glycoprotein is an antibody.

16. A glycoprotein obtainable by the method of claim 1.

17. A higher plant cell glycoprotein having a complex N-glycan profile devoid of β(1,2) xylosyl and α (1,3) fucosyl and further comprising terminally linked β(1,4) galactosyl residues.

18. The glycoprotein according to claim 17, wherein a β(1,4) galactosyl residue has been transferred to at least 30% of the terminally linked N-acetylglucosamine residues.

19. A cell of a higher plant comprising a reduced level of β(1,2) xylosyltransferase and α (1,3) fucosyltransferase activity and an functional β(1,4) galactosyltransferase activity.

20. The plant cell according to claim 19, comprising no β(1,2) xylosyltransferase and α (1,3) fucosyltransferase activity.

21. The plant cell according to claim 19, comprising a chimeric gene comprising the following operably linked DNA regions:
i. a plant-expressible promoter
ii. a DNA region encoding said β(1,4) galactosyltransferase; and
iii. a DNA region involved in transcription termination and polyadenylation.

22. The plant cell according to claim 20, wherein said β(1,4) galactosyltransferase activity is a mammalian β(1,4) galactosyltransferase.

23. The plant cell according to claim 20, wherein said mammalian β(1,4) galactosyltransferase is a human β(1,4) galactosyltransferase.

24. The plant cell according to claim 20, wherein said β(1,4) galactosyltransferase activity is a hybrid β(1,4) galactosyltransferase activity.

25. The plant cell according to claim 20, wherein said reduced level of β(1,2) xylosyltransferase and α (1,3) fucosyltransferase activity is the result of a null mutation in the endogenous β(1,2) xylosyltransferase and α (1,3) fucosyltransferase encoding gene.

26. The plant cell according to claim 20, wherein said reduced level of β(1,2) xylosyltransferase and α (1,3) fucosyltransferase activity is achieved by transcriptional or post-transcriptional silencing of the expression of endogenous β(1,2) xylosyltransferase and α (1,3) fucosyltransferase encoding gene.

27. The plant cell according to claim 20, further comprising a glycoprotein foreign to said higher plant cell.

28. The plant cell according to claim 27, wherein said glycoprotein is expressed from a chimeric gene comprising a plant expressible promoter operably linked to a coding region encoding said glycoprotein.

29. The plant cell according to claim 20, wherein said glycoprotein is a mammalian protein.

30. The plant cell according to claim 20, wherein said glycoprotein is a therapeutic protein.

31. The plant cell according to claim 20, wherein said glycoprotein is an antibody.

32. A higher plant consisting essentially of the plant cells according to claim 20.

33. A method to modify the N-glycosylation pattern of glycoproteins in higher plant cells, said method comprising the step of generating a plant cell comprising a reduced level of β(1,2) xylosyltransferase and α (1,3) fucosyltransferase activity and a functional β(1,4) galactosyltransferase activity.

34. (canceled)

35. The method according to claim 9, wherein said DNA region encoding said β(1,4) galactosyltransferase comprises the nucleotide sequence of SEQ ID No 10 from nucleotide position 523 to nucleotide position 1719.


SEQUENCE LISTING (14 sequences)

SEQ ID NO: 1 - DNA, 21 bp, Artificial
  FucTA Left primer (LP)
tgatccccttgacttgctacc

SEQ ID NO: 2 - DNA, 21 bp, Artificial
  FucTA Right primer (RP)
atgccacaacttagcatctcc

SEQ ID NO: 3 - DNA, 26 bp, Artificial
  FucTB Left primer (LP)
ttctgtgtaatatttttgttcgttgg

SEQ ID NO: 4 - DNA, 21 bp, Artificial
  FucTB Right  primer (LP)
caaggcacttgttgatatggc

SEQ ID NO: 5 - DNA, 21 bp, Artificial
  XylT Left primer (LP)
tggagattgtttttgagtcgg

SEQ ID NO: 6 - DNA, 21 bp, Artificial
  XylT Right primer (LP)
ggaagctcctcctcctctttc

SEQ ID NO: 7 - DNA, 22 bp, Artificial
  Left Border T-DNA primer (LP)
gcgtggaccgcttgctgcaact

SEQ ID NO: 8 - DNA, 28 bp, Artificial
  Forward huGalT primer
caactcatgaggcttcgggagccgctcc

SEQ ID NO: 9 - DNA, 29 bp, Artificial
  Reverse huGalT primer
caacaagcttctagctcggtgtcccgatg

SEQ ID NO: 10 - DNA, 1949 bp, Artificial
  plant-expressible huGalT chimeric gene
tttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactcgagctcatttctctattacttcagccataacaaaagaactcttttctcttcttattaaaccaaaaccatgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctagaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatc

SEQ ID NO: 11 - protein, 365 aa, Artificial
  huGalT
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIDIGTPS

SEQ ID NO: 12 - DNA, 2045 bp, Artificial
  XylT gene Arabidopsis thaliana
aaatctgcagactctcaaaattccgattcatcttattgaagaacaattttccggcgaaacagccgatgaagtctcgcctgaatcttctgtacctttcaccggcgattgacttcacttcagaatcgagagagaagaaatcgatggaaaactaaaaatagaaagagtttcaaattctcgctctctcttcaaaaccgcaaatcaagggaacgagagacgagagagagagatgagtaaacggaatccgaagattctgaagatttttctgtatatgttacttctcaactctctctttctcatcatctacttcgtttttcactcatcgtcgttttcaccggagcagtcacagcctcctcatatataccacgtttcagtgaataaccaatcggcgattcagaaaccgtggccgatcttaccttcttacctcccatggacgccgccgcagaggaatctaccaactggctcctgcgaaggttacttcgggaatggatttacaaagagagttgacttccttaagccgaggattggaggaggaggagaaggaagctggttccgatgtttttacagtgagacattacagagttcgatttgtgaaggaaggaatctgagaatggttccggatcggattgttatgtcgagaggaggtgagaagttagaggaagttatggggaggaaagaggaggaggagcttcctgcgtttcgacaaggtgcgtttgaggtagcggaagaggtttcttcacggttaggttttaagagacaccgtcgttttggtggaggagaaggaggtagtgcggtttctcggcggctggtgaatgatgagatgttgaatgaatatatgcaagaaggtggaattgatagacatacaatgagagatttggttgcttcgattcgtgctgttgataccaatgatttcgtttgtgaagagtgggtggaggaaccgaccttgcttgtcactagattcgagtacgcaaatctcttccatactgtgacagattggtatagtgcctatgtttcgtctagagtcaccggtttgcctaatcgacctcacgttgttttcgttgacggacactgcacgacgcagctagaagaaacatggacagctttgttttccggaatcagatacgcaaagaacttcaccaaaccggtttgtttccgccacgcgattctttcaccattgggatacgaaaccgctctttttaaaggcttgtccggagaaatagactgcaagggagattcagctcacaatctgtggcaaaacccggacgataaaaggactgcgaggatatcagagtttggtgaaatgatcagagcagctttcgggttgcctgtcaatagacaccgctcattagaaaagccgctatcatcatcatcatcatcagcctcagtttataatgttctttttgtccgccgtgaagattacttagcccatcctcgtcatggcggtaaagtccagtctcggctcatcaatgaggaagaagtgttcgactcgttgcatcattgggttgcaactgggtccaccggtctgaccaaatgcgggattaaccttgtgaatggcttgcttgcacacatgtcaatgaaagatcaagtccgagccattcaagatgcttcagtgatcataggagctcatggagcaggactgactcacattgtctctgcaacaccaaacacaacgatatttgagataataagcgtcgagtttcagagacctcatttcgagcttatagctaagtggaaaggattggagtatcacgcgatgcatctggcgaactcacgagcggaaccaacggctgtgattgagaagttaacggagatcatgaagagccttggctgctaaagacaaaagaaaaaacaagaatttaggatagtgagtgagttctaatctccggtgaccggtgagtttgtcatattttgataccttgtatttttgtatcattttttcgatttggttaatgatgtaaatttggcttttattttttgtactaaacatgatttctaatggttatcttatctattattattttacaattcctttttaaaaggaaaatgcc

SEQ ID NO: 13 - DNA, 1810 bp, Artificial
  FucTA gene Arabidopsis thaliana
ctttctcatcaatcaaagtatcaaacgataaaaacccaaatcacaattcttaaaatccattcattattgataaaaaatcgtcgctttgataatgggtgttttctccaatcttcgaggtcctaaaattggattgacccatgaagaattgcctgtagtagccaatggctctacttcttcttcttcgtctccttcctctttcaagcgtaaagtctcgacctttttgccaatctgcgtggctcttgtcgtcattatcgagatcgggttcctctgtcggctcgataacgcttctttggtcgatacgttaacccattttttcaccaagtcgtcgtccgatttgaaagttgggtcaggaatagagaaatgccaggagtggttagagagagtggattcagttacttattctagagatttcactaaagatccgatttttatctctggtagtaacaaggacttcaaatcgtgctctgttgattgtgtaatgggattcacttcagataagaaacctgatgcggcttttggattaagtcatcaacctggaacactcagtataatccgttccatggaatcagcacagtattaccaagagaataatcttgctcaagcacgacggaaaggttatgatattgtgatgacaactagtctgtcatcagatgttcctgttgggtatttttcatgggcggaatatgatattatggctccagtgcaaccaaaaacagagaaagctcttgctgctgcttttatttccaattgcgccgctcggaatttccgcctgcaagctcttgaagccttaatgaagacgaatgttaagattgattcttatggtggttgtcaccggaatcgggatgggagtgtggagaaggttgaagctcttaagcactacaaattcagtctagcttttgagaacaccaacgaggaggattatgtcacagagaagttcttccaatctctagtcgctggatctgtccctgtggttgttggagctccaaatatagaagaatttgcaccttctcctgactcattccttcacattaagcagatggatgatgtcaaggcagttgcaaagaaaatgaagtatcttgcggataaccctgacgcctataatcagacgctaagatggaaacatgaaggcccttcagattcttttaaggcacttattgatatggctgctgtacactcttcttgtcgtctctgcatctttgtggctacaaggattcgtgagcaagaagagaagagccctgagtttaagagacgaccctgcaaatgcaccagaggctcagagacagtttatcatttgtatgttagagaaagaggacggtttgacatggaatccatcttcttgaaggatggaaatctgactctggaagctctggaatctgcggttcttgcgaagttcatgtctctgagatatgaaccaatatggaagaaggaaagacccgcgagcttaagaggagacggcaagcttagagtacatgggatatatcctattggtctgactcaaagacaagctctttacaacttcaaattcgaaggaaattcaagtctcagtactcacatacagagaaacccttgtcccaaattcgaagttgtctttgtctaaattctagaagaaaaccaaagtttattttgtgatacatgctttgagtgtagtttgtcttaggcaggaattaaggaatgtgtacatataaaaataaaagagtttttgcttgtcttattgggtactacaatgcacatatgttcaagtgtagtttgataaaacacaaaatgacacaagcattctcagattagctttaacagatttacagatactgca

SEQ ID NO: 14 - DNA, 1985 bp, Artificial
  FucTB gene Arabidopsis thaliana
taaacttaataaagcctcgtactgagagatcaaaacaaaacaaaacaaaacccaaacacttaccaaatcaatcaattatcgagaatcttccttcctttaatcctcaaaaaaaacaaaaacctttcttcacctcctttccttgattcatcctctaggttaatgggtgttttctcgaatcttcgaggacccagagccggagctacccacgatgaatttccggcgaccaatggctctccttcgtcttcttcttctccatcttcatcaatcaagcgaaaattatcgaatttgttaccactctgcgttgctctggtagttatcgctgagatcgggtttctgggtcggctcgataaagtcgctttggttgatacgttgactgatttcttcacccagtctccgtcactctcgcagtctccaccggcgagatccgatcggaagaagatcggattatttactgataggagctgcgaggagtggttgatgagagaagattcagttacttactctagagattttactaaagatccaatttttatctctggtggtgaaaaggactttcaatggtgttctgtggattgtacatttggagatagttcagggaaaacaccagatgctgcgtttggattaggtcagaaacctggaactcttagtataatacgttccatggaatcagcacagtattatccagaaaatgatcttgcacaggcacgacggagaggttatgatatagtgatgaccactagtctatcatcagatgttcctgttggatatttttcgtgggcggagtatgatattatgtctccggtacagccaaaaactgagagagctattgcagctgcttttatttctaattgtggtgctcggaattttcgtctacaagcacttgaggcattgatgaaaactaacattaagattgattcttatggtggttgtcatcgaaaccgggatgggaaagttgacaaggttgaagctcttaagcgatacaaattcagtttggcttttgagaatactaacgaggaagattatgtcaccgagaagttctttcaatccttagttgctgggtccgtccccgtggtagttggtcctccaaatatagaagaatttgcgcctgcttcggactcattccttcacattaagactatggaagatgtagagccagttgcaaagagaatgaagtatctcgcagctaaccctgctgcttataatcagacactaagatggaaatacgagggtccttcagattctttcaaggcacttgttgatatggctgctgtacactcttcttgccgtctctgcattttcctggccacgagggtccgagaacaagaagaggaaagccctaatttcaagaaacgaccgtgcaaatgtagcaggggaggatcagacacagtttatcatgtttttgttagagaaagaggccggtttgaaatggaatcagtctttttgaggggtaaaagtgtgactcaggaagctctagaatctgcagttctcgccaagttcaagtctttaaaacatgaggcagtgtggaagaaggaaaggcctggaaacttaaaaggagacaaagagcttaaaatacatcggatttacccgcttggcctaacgcaacgacaggctttgtacaacttcaaattcgagggaaattcgagtctaagtagtcacattcaaaacaacccttgtgctaaatttgaggttgtcttcgtctagtttcattcctctggatctgtcacaggtatcatctcagctaagaagacatttctctgtgctagaatcgcaaagtgctaaacaaaccgattagatgaaacaaaaggttaatagtcatgagattggtgaactcattttgtttaggcagtgtatctgtaaatcgttctgacattgcagacgatgtgttcttgatagctggatgcataaatgtttgaagatttagagcaatttgatagttttgaatctcttgagagtgtgttaattaatctttaaatttttcttgggtt